(19) United States
(12) Patent Application Publication
(10) Pub. No.: US 2010/0249931 A1
(43) Pub. Date: Sep. 30, 2010

(54) MECHANICALLY COMPETENT NATURAL POLYMER BASED POROUS GRAFTS FOR BONE REPAIR AND REGENERATION

(75) Inventors: Cato T. Laurencin, Avon, CT (US); Sangamesh G. Kumbar, South Windsor, CT (US); Syam Prasad Nukavarapu, New Britain, CT (US); Roshan James, Charlottesville, VA (US)

Correspondence Address: CANTOR COLBURN, LLP, 20 Church Street, 22nd Floor, Hartford, CT, 06103, US

(73) Assignees: UNIVERSITY OF CONNECTICUT, Farmington, CT (US); University of Virginia, Charlottesville, VA (US)

(21) Appl. No.: 12/710,637

(22) Filed: Feb. 23, 2010

Related U.S. Application Data
(60) Provisional application No. 61/154,582, filed on Feb. 23, 2009.

(51) Int. Cl. A61F 2/28

(52) U.S. Cl. 623/16.11

(57) ABSTRACT

The invention provides a scaffold for bone or cartilage replacement, in which the scaffold is fabricated from naturally derived polymers. In one embodiment the invention provides a bone replacement scaffold, wherein the scaffold comprises sintered polysaccharide microspheres. In a particular embodiment the invention provides a bone replacement scaffold, in which the scaffold comprises polysaccharide microspheres comprising ethyl cellulose microspheres and/or cellulose acetate microspheres. The invention further includes methods of making bone replacement scaffolds and methods of treating bone injury in an animal.

### CROSS REFERENCE TO RELATED APPLICATION

This application claims priority from U.S. Provisional application No. 61/154,582 filed Feb. 23, 2009, which is hereby incorporated by reference in its entirety.

### FIELD OF THE INVENTION

A bone replacement scaffold fabricated from naturally derived polymers is provided herein. Methods of making bone replacement scaffolds are also provided herein.

### BACKGROUND

The repair and replacement of damaged hard tissues such as bone is a major clinical problem in the U.S. and around the world. In the U.S. alone, more than 500,000 hip and knee replacements are performed and over a million fractures are treated each year (Bucholz, Clin. Orthop. (2002) 398: 44-52). These numbers are expected to grow as the US population grows and the life expectancy of the population increases. Current bone replacement procedures often use autograft or allograft tissue but these approaches have limitations. Autograft tissue is often limited in supply and carries the potential for donor site morbidity. Allograft tissue carries the potential for disease transmission and immunological rejection (Mankin, Clin. Orthop. Relat. Res. (2005) 432: 210-216).

The field of tissue engineering seeks to design tissue substitutes for clinical use to replace diseased organs or to heal and regenerate damaged tissue. The tissue engineering approach holds potential for overcoming the limitations associated with the use of autografts and allografts. Scaffold based tissue engineering has become a promising strategy to regenerate three-dimensional (3-D) tissues for transplantation. In the scaffold approach a three dimensional framework, or scaffold, is constructed and inserted at the tissue damage site. The scaffold then provides a surface for the attachment and re-growth of biological tissue. A three-dimensional bioresorbable porous construct with appropriate mechanical properties is required to guide cellular attachment and subsequent tissue formation (Borden, et al., Biomaterials, (2002) 23: 551-559; Katti and Laurencin, in Advanced Polymeric Biomaterials, Shonaike and Advani (eds.) CRC Press, Boca Raton, 2005, 484-527; Uhrich, et al., Macromolecules, (1995) 28: 2184-2193; Kumbar, et al., J. Inorg. Organometallic Polym. Mater. (2006) 16: 365-385; and Kofron, et al., J. Biomed. Mater. Res. A. (2007) 82: 415-425).

A wide range of synthetic and natural polymers have already been adopted for 3-D scaffold fabrication. Synthetic biodegradable polymers such as poly(esters), poly(anhydrides), poly(anhydride-co-imides) and poly(phosphazene) derivatives, have been used to fabricate scaffolds for bone repair. These synthetic materials have been investigated as potential candidates for scaffold fabrication due to their programmable degradation characteristics (Laurencin, et al., in Annual Review of Biomedical Engineering, Yarmush (ed.) Annual Reviews Inc., Palo Alto, (1999) 1: 19-46). The α-hydroxyesters poly(lactic acid) (PLA), poly(glycolic acid) (PGA) and their copolymer PLAGA are approved by the Food and Drug Administration (FDA) for certain biomedical applications (Athanasiou, et al., Arthroscopy, (1998) 14: 726-737).

The utility of synthetic scaffold materials in transient biomedical applications, including implants, is hampered due to the acidic degradation products that can adversely affect the biocompatibility (Taylor, et al., J. Appl. Biomaterials, (1994) δ: 151-157). This problem becomes more acute in larger sized implants or at implant sites with minimal fluid flow. For instance, in articular cartilage the acidic degradation products can accumulate significantly and affect the cells and the tissues surrounding the implant. A clinical study involving 1000 patients over a period of 9 years reported that a significant number of patients developed inflammatory foreign body reaction to implants that were made of PGA, PLA and PLAGA. Some of the patients developed severe osteoarthritis in the joints near the implants and some of them had to undergo arthrodeses (Bostman, et al., J. Bone Joint Surg., (1998) 80: 333-338). These drawbacks with the hydroxyesters underscore the need for new polymers for biomedical applications that degrade into non-toxic and non-immunogenic byproducts.

Scaffolds derived from the polymers of natural origin have shown superior biological performance due to their chemical similarity with the extracellular matrix (ECM) components, which the biological environment is prepared to recognize and deal with metabolically. Natural polymer scaffolds may also avoid the stimulation of chronic inflammation or immunological reactions and toxicity often associated with synthetic polymer scaffolds. However, the currently available scaffolds fabricated from natural origin material do not possess adequate mechanical properties, interconnected pore structure and/or porosity for bone healing applications at load bearing sites. Thus there remains a need for new polymer scaffolds useful for bone healing and regeneration. The bone replacement and repair scaffolds provided herein fulfill this need and provides additional advantages described herein.

### SUMMARY OF THE INVENTION

A bone replacement scaffold is provided herein, in which the scaffold is fabricated from naturally derived polymers. In one embodiment the invention provides a bone replacement scaffold, wherein the scaffold comprises sintered polysaccharide microspheres. In a particular embodiment the invention provides a bone replacement scaffold, in which the scaffold comprises polysaccharide microspheres comprising ethyl cellulose microspheres and/or cellulose acetate microspheres.

A method of making a bone replacement scaffold is provided herein, in which the scaffold is fabricated from naturally derived polymers.

A method of making a bone replacement scaffold is provided herein comprising: providing a plurality of polysaccharide microspheres; providing a solvent system having an organic solvent fraction and an aqueous fraction; mixing the polysaccharide microspheres and the solvent system to form a slurry; molding the slurry to form a scaffold; and removing the organic solvent fraction from the scaffold. In a certain embodiment the polysaccharide microspheres include ethyl cellulose microspheres and/or cellulose acetate microspheres.

A method of making a bone replacement scaffold is provided herein comprising: providing a plurality of polysaccharide microspheres; providing a solvent system having an organic solvent fraction and an aqueous fraction; mixing the polysaccharide microspheres and the solvent system to form a slurry; molding the slurry to form a scaffold; removing the organic solvent fraction from the scaffold; and incubating the scaffold with a collagen solution after removal of the organic solvent fraction from the scaffold.

### BRIEF DESCRIPTION OF THE DRAWINGS

FIG. 1. Morphology of porous 3-D microsphere scaffolds (A-D).

FIG. 2. Morphology of porous uncoated Cellulose Acetate scaffolds (A-B) and Cellulose Acetate scaffolds coated with collagen nanonfibers (C-F).

FIG. 3. Morphology of porous uncoated Cellulose Acetate scaffolds coated with collagen nanonfibers (G-I).

FIG. 4. Morphology of porous Ethyl Cellulose scaffolds without collagen nanofiber functionalization at lower (A) and higher (B) magnification. Ethyl Cellulose scaffolds coated with collagen nanonfibers (C-F).

FIG. 5. Morphology of porous Ethyl Cellulose scaffolds with collagen nanofiber functionalization (G-L). Scaffolds in FIGS. 1-5 are fabricated using the solvent/non-solvent sintering method.

FIG. 6. Mechanical properties of microsphere scaffolds. Variation of Compressive modulus (A) and Compressive strength (B) with microsphere diameter.

FIG. 7. Mechanical Properties of Cellulose Acetate Porous Grafts, molecular weight 50,000. Graphs represent (A) Maximum Load (N) grafts can withstand, (B) Compressive modulus (MPa), (C) Compressive Strength (MPa). (D) Energy at Failure (J) and Maximum Compressive Load (N).

FIG. 8. The mechanical properties of cellulose acetate porous grafts of molecular weight 30,000.

FIG. 9. Mechanical Properties of Ethyl Cellulose Porous Grafts, molecular weight 30,000.

FIG. 10. Degradation of CA and EC scaffolds over a 10-week period. Both scaffolds showed a progressive decrease in average molecular weight with time.

FIG. 11. Degradation of porous grafts of CA and EC conducted at 37° C. in phosphate buffer at pH 7.4 in presence and absence of Cellulase enzyme over 6 months time period.

FIG. 12. Mechanical Performance of Cellulose Acetate Porous Grafts, molecular weight 30,000 over period of 24 weeks of degradation in Phosphate Buffer pH 7.4 at 37° C.

FIG. 13. Mechanical Performance of Cellulose Acetate Porous Grafts, molecular weight 30,000 over period of 24 weeks of degradation in Cellulase Enzyme (2 wt/vol % in Phosphate Buffer pH 7.4) at 37° C.

FIG. 14. Mechanical Performance of Ethyl Cellulose Porous Grafts, molecular weight 30,000 over period of 24 weeks of degradation in Phosphate Buffer pH 7.4 at 37° C.

FIG. 15. Mechanical Performance of Ethyl Cellulose Porous Grafts, molecular weight 30,000 over period of 24 weeks of degradation in Cellulase Enzyme (2 wt/vol % in Phosphate Buffer pH 7.4) at 37° C.

FIG. 16. Gentamcin release over time from 15% and 5% drug-loaded scaffolds.

FIG. 17. MC3T3E1 preosteoblast proliferation on EC and CA scaffolds showed steady growth, which is expressed as DNA (ng/ml) per sample. * indicates statistical significance, p<0.05, within the same time point. Control PLAGA microsphere scaffolds had significantly higher DNA amounts at all the time points.

FIG. 18. Expression of alkaline phosphatase by MC3T3E1 preosteoblasts on polysaccharide scaffolds expressed as units of ALP per pg of DNA. * indicates statistical significance, p<0.05, within the same time point. Cells expressed higher levels of ALP on polysaccharide scaffolds up to 14 days indicating early cell differentiation on cellulose scaffolds as compared to PLAGA control scaffolds.

FIG. 19. Alizarin Red stained scaffolds (A) CA, (B) PLAGA and (C) EC (top panel) cultured in osteogenic media after 7 days demonstrates scaffold mineralization. Bottom panel respective control scaffolds without cells.

FIG. 20. Confocal images (A-B): Cell survival and morphology of MC3T3E1 seeded on polysaccharide scaffolds on day 7 as determined by viability/cytotoxicity assay at 10× magnification.

FIG. 21. (A) Heat sintered PLAGA (B) CA and EC solvent/non-solvent sintered 3-D microsphere scaffolds closely mimic the structure of (C) native bone. PLAGA scaffolds were evaluated in a (D) critical size New Zealand White rabbit ulnar defect model and (E) 3-D micro-CT reconstructions at 24 weeks post-implantation anterior and posterior views of the PLAGA scaffold. Histology images (F and G) 24 weeks post-implantation showed robust osteoid (stained blue) formation and mineralized tissue (stained red). More than half of the PLAGA scaffold was not mineralized (G) presumably due to acidic degradation products inhibiting mineralization in portions of the implant. Polysaccharide microsphere scaffolds (B) avoid acidic degradation issues due to acidic degradation products and improve mineralization and accelerate bone healing.

### DETAILED DESCRIPTION

### Terminology

The following terminology may be helpful before considering the detailed description of the invention, which follows.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

In all occurrences where the word “about” appears with a range, e.g. “about 550 micrometers to about 1200 micrometers” the exact range is also included, in which the word about does not appear. Thus the invention also pertains to microspheres of 550 micrometers to 1200 micrometers.

The word “comprising” appears the language is meant to be open-end as is it commonly understood to be in patent claims; the inclusion of additional elements is contemplated. In all occurrences where the word “comprising” is used the invention also includes embodiments in which the less open language “consisting essentially of” or “consisting of” can be used.

“Derivatized cellulose” is cellulose that has been chemically modified, either naturally or synthetically. Derivatized cellulose, as used herein, is a polysaccharide derivative. Derivatized cellulose includes, but is limited to methyl cellulose, ethyl cellulose, carboxy methylcellulose, hydroxyethyl cellulose, hydroxypropyl methylcellulose, ethyl methylcellulose, etc. and cellulose acetate.

“Functionalized with collagen nanofibers” means collagen nanofibers are added to the scaffold or microspheres thereby providing additional surface area on the scaffold or microspheres respectively.

“Polysaccharides” are polymers comprised of many monosaccharides joined together by glycosidic bonds. As used herein “polysaccharides” include both natural polysaccharides, such as cellulose and chitin, and synthetic polysaccharide derivatives, such as derivatized cellulose.

“Bone replacement” includes, in certain embodiments, temporary and permanent replacement, as well as providing a repair scaffold for bone regeneration.

“Sintering” is the thermal treatment of a powder or compact at a temperature below the melting point of the main constituent, for the purpose of increasing its strength by bonding together of the particles. “Sintered” materials are any materials that have been formed by the process of sintering.

A “Solvent/non-solvent composition” is a solvent system having at least two fractions—a volatile organic fraction (the solvent) and a non-volatile, typically aqueous, fraction. A preferred embodiment is a solvent/non-solvent composition having an organic solvent fraction and an aqueous (non-solvent) fraction. Appropriate solvent fractions include, but are not limited to, acetonitrile, acetone, hexanes, dichloromethylene, methanol, ethanol, and methylethylketone. Solvent/non-solvent compositions include acetone:water (e.g. 3:1) and acetonitrile:water (e.g. 8:1).

### Bone Replacement Scaffolds

A microsphere scaffold system is provided herein, which combines the biocompatibility of natural polymers with a novel scaffold structure, having adequate mechanical properties for bone healing applications at load bearing sites. It is possible to alter mechanical properties of the microsphere scaffold, including, porosity and degradation profile, by altering the polymer molecular weight, using different polysaccharide derivatives, and by varying the microsphere composition. With the solvent/non-solvent sintering method provided herein scaffolds of virtually any size and shape can be fabricated. Microspheres, which incorporate growth factors or antibiotics to accelerate bone healing, are also provided. Surfaces of the microsphere scaffolds functionalized with collagen nanofibers provide enhanced surface area, promote cell attachment, and favor matrix mineralization in vivo. Included herein are any embodiments as described herein in which the scaffold for bone or cartilage replacement additionally comprises an antibiotic, a growth factor, a tissue response modifier, or collagen. In certain embodiments the collagen is in the form of collagen nanofibers, is collagen type I, and/or is added to the scaffold for bone or cartilage replacement at a 0.5% to 2.0% w/v collagen solution. Also included herein are any embodiments as described herein in which the scaffold for bone or cartilage replacement is functionalized with collagen nanofibers.

The combination of collagen nanofibers and microsphere scaffolds is useful for delivery combinations of growth factors that may be released simultaneously or sequentially during bone healing. Since scaffold fabrication can be achieved at a temperature close to the physiology it is possible to incorporate growth factors and antibiotics during scaffold fabrication without altering their bioactivity. Additionally multiple factors can be released in a sequential manner using the composite structure. For instance functionalized nanofibers can release angiogenic factor (VEGF growth factor) in the beginning to promote scaffold vascularization, while microspheres can contribute a delayed release of osteogenic factor (BMP-2 growth factor) through diffusion and erosion mechanisms, to promote osteogenesis.

A bone replacement scaffold comprising sintered microspheres in which the microspheres are polysaccharide microspheres is provided. In certain embodiments the microspheres are cellulose acetate microspheres, ethylcellulose microspheres or a combination thereof. In certain embodiments the polysaccharide microspheres have a microsphere diameter of about 100 micrometers to about 1200 micrometers, or of about 1180 μm, about 1180 to about 850 μm, of about 850 to about 600 μm, of about 300 to about 650 micrometers, and in a particular embodiment of about 650 to about 850 micrometers. In certain embodiments the scaffold comprises at least 70 percent by weight sintered polysaccharide microspheres. In certain embodiments the sintered polysaccharide microspheres are ethyl cellulose or cellulose acetate microspheres. Included herein are bone replacement scaffolds formed into a shape suitable for administration to bone. For example, the bone replacement scaffolds comprising sintered polysaccharide microspheres formed into the shape of a missing bone section are included herein.

Mechanically competent, porous, 3-D sintered microsphere scaffolds comprised of naturally derived polymers, which in certain embodiments are also functionalized with nanofiber structures of natural origin are provided. Microspheres of polysaccharides, for example, cellulose acetate (CA) or ethyl cellulose (EC), are sintered together into a 3-D scaffold using a novel solvent/non-solvent approach. Biomemitic microsphere scaffolds are created though the addition of collagen nanofibers on the surface of microspheres. In certain embodiments the microsphere scaffolds provided herein are functionalized on their surfaces with collagen nanofibers. The collagen nanofibers provide topography, which improves the biological functioning of the microsphere scaffolds.

The microsphere scaffolds provided herein are sintered together in into 3-D structures using a novel solvent/non-solvent sintering approach. As such, methods for making 3-D sintered microsphere scaffolds comprised of naturally derived polymers are also provided.

The novel composite scaffold provided herein has a hierarchical design composed of macro-, micro- and nanostructures that have shown to accelerate tissue regeneration. Scaffold 3-D architecture (macro structure) provides mechanical stability; individual microspheres (micro structure) sintering provide adequate porosity and nanofibers provide nanotopography. Solvent/non-solvent microsphere sintering avoids the elevated temperature during scaffold fabrication and thus prevents heat mediated denaturing of growth factors or drugs loaded in the microspheres. Scaffolds fabricated on the proposed platform are reproducible, scalable and offer great control over pore size, porosity and mechanical properties. Additionally these scaffolds are cost effective when compared to synthetic polymers.

Many of the bone replacement scaffolds comprised of sintered microspheres of naturally derived polymers are autoclavable. That is they are able to withstand heating in a pressurized device, typically at 121 degrees C. for 15 minutes or at 134 degrees C. for 3 minutes, without significant deterioration. This is an important advantage as materials implanted into living bone must be infection free. Certain of the bone replacement scaffolds disclosed herein are also able to withstand heating up to 200 degrees C. for 10 minutes at atmospheric pressure. Included in the invention are any embodiments described herein in which the bone replacement scaffold is autoclavable or autoclaved.

Sintered microsphere scaffolds comprised of synthetic polymers for bone tissue engineering application have previously been disclosed. See U.S. Pat. No. 5,866,155 which is hereby incorporated by reference for its disclosure regarding sintered microsphere scaffolds. Sintered microsphere scaffolds comprised of natural polymers such as polysaccharides, for example CA or EC, which are useful for bone tissue engineering applications at load bearing sites are provided herein. The materials provided herein are useful for bone healing applications. Microspheres having surfaces functionalized with collagen nanofibers, which microspheres provide enhanced surface areas for cell attachment and growth are provided.

The invention provides a mechanically competent composite microsphere scaffold system comprised of naturally derived polymers which microsphere scaffold system is functionalized with nanofibers. The naturally derived polymers include polysaccharides and the nanofibers include collagen nanofibers. The naturally derived composite 3-D sintered microsphere scaffolds provide adequate mechanical properties, porosity and surface nanotopography for accelerated bone healing at load bearing sites.

The present invention also provides microsphere scaffolds comprised of naturally derived polymers, which scaffolds show improved osteoblast adhesion, differentiation proliferation, and phenotype expression over synthetic microsphere scaffolds, for example polyester scaffolds. The invention also provides microsphere scaffold having improved deposition of a mineralized matrix of primary human osteoblast cells cultured on composite microsphere scaffolds as compared to primary human osteoblast cells cultured on synthetic microsphere scaffolds.

A majority of the synthetic grafts and fixation devices are polyester based and found to stimulate chronic inflammation and foreign body reaction. The inventors have surprisingly discovered that natural polymer cellulose can be processed into fixation devices with adequate mechanical properties for long-term implantation applications. Cellulose derivatives can provide tunable degradation properties for transient scaffolding applications. Furthermore scaffolds can be sterilized by autoclaving, which is desirable in clinical applications, without compromising their mechanical properties.

The invention also provides microsphere scaffolds comprised of naturally derived polymers useful for bone healing, which microsphere scaffolds elicit less inflammatory response than microsphere scaffolds derived from synthetic polymers, for example polyester scaffolds. In a preferred embodiment the invention provides microsphere scaffolds comprised of naturally derived polymers useful for bone healing, which do not stimulate any clinically significant inflammatory response.

As stated above the invention provides microsphere scaffolds useful for bone replacement and comprised of naturally derived polymers, which additionally include growth factors, tissue response modifiers, and/or antibiotics. Growth factors suitable for use with the microsphere scaffold of the invention include human growth hormone, or in the instance of veterinary applications animal growth hormones, such as bovine growth hormone. Other suitable growth factors include bone morphogenic factors; such as members of the transforming growth factor beta family and other bone morphogenic factors include BMP8a, BMP8b, and BMP10 to BMP15. Suitable tissue response modifiers include VEGF.

Antibiotics suitable for use with the microsphere scaffold of the invention include but are not limited to penicillins, cephalosporins, carbacephems, cephamycins, carbapenems, monobactams, aminoglycosides, glycopeptides, quinolones, tetracyclines, macrolides, and fluoroquinolones. Use of antibiotics prescribed for osteomyelitis with the microsphere scaffold of the invention is particularly contemplated as well as the use of antibiotics use for treating Staphylococcus Aureas and Methicillin Resistant Staphylococcus Aureas infections. Antibiotics that may be used with the microsphere scaffold of the invention include but are not limited to penicillin G; methicillin; nafcillin; oxacillin; cloxacillin; dicloxacillin; ampicillin; amoxicillin; ticarcillin; carbenicillin; mezlocillin; azlocillin; piperacillin; imipenem; aztreonam; cephalothin; bacitracin; cefazolin; cefaclor; cefamandole formate sodium; cefoxitin; cefuroxime; cefonicid; cefmetazole; cefotetan; cefprozil; loracarbef; cefetamet; cefoperazone; cefotaxime; ceftizoxime; ceftriaxone; ceftazidime; cefepime; cefixime; cefpodoxime; cefsulodin; fleroxacin; nalidixic acid; norfloxacin; ciprofloxacin; Ofloxacin; Enoxacin; lomefloxacin; cinoxacin; doxycycline; minocycline; tetracycline; amikacin; gentamycin; kanamycin; netilmicin; tobramycin; streptomycin; azithromycin; clarithromycin; erythromycin; erythromycin estolate; erythromycin ethyl succinate; erythromycin glucoheptonate; erythromycin lactobionate; erythromycin stearate; vancomycin; teicoplanin; chloramphenicol; clindamycin; trimethoprim; sulfamethoxazole; nitrofurantoin; rifampin; mupirocin; metronidazole; cephalexin; roxithromycin; co-amoxiclavuanate; piperacillin and tazobactam; and any combination of the foregoing, including their various salts, acids, bases, and other derivatives.

The microsphere scaffolds of the invention may also include enzymes, such as cellulase. In this embodiment the cellulase is present in a controlled release form so that the cellulase is released and slowly degrades the microsphere scaffold after scaffold implantation has occurred and bone healing has begun. Using a controlled release cellulase preparation, the decay rate of the implanted microsphere scaffold may be matched to the rate of bone repair.

### General Methods

Porous 3-D composite microsphere scaffolds, comprised of naturally derived polymers, are fabricated with a target pore size range provide physical characteristics suitable for a bone regeneration scaffold. Polysaccharides microspheres, for example CA or EC microspheres, are produced by an oil-in-water emulsion/solvent evaporation method. In this method 4 g of EC is dissolved in 18 mL of a solvent mixture of methylene chloride and acetone at a ratio 9:1. The resulting polymer solution is emulsified by pouring into a 1% polyvinyl alcohol solution and stirring at 250 rpm. Stirring is maintained under atmospheric pressure and room temperature for 24 h to allow complete evaporation of the organic solvent. The microspheres are isolated, washed with deionized water, dried, and sieved. Individual microspheres of different diameters namely >1180, 1180-850, 850-600, 800-710, 710-600, 600-500 425-300, 400-300, and 300-200 μm are used for fabricating 3-D sintered microsphere scaffolds. Individual microspheres of about 200 to 1200 μm diameter, or in certain embodiments about 500 to about 800 μm diameter, are useful for fabricating 3-D sintered microsphere scaffolds The organic solvent is removed. In some instances the solvents form an azeotrope, which is removed under vacuum.

Polysaccharide microspheres may also be created by electrospraying the cellulose derivatives followed by solution drying. Microsphere shapes may be adjusted by adjusting the conditions by which the microspheres are created. Typically spherical shapes are preferred, though elongated shapes may be useful for some applications. Adjusting microsphere size and shape is a matter of routine experimentation, readily ascertained by those of skill in the art of creating polysaccharide microspheres.

Microspheres, e.g. EC microspheres, of a chosen diameter range are mixed with a solvent/non-solvent composition of 3:1 ratio of acetone:water to produce a slurry. The resulting slurry is placed in a cylindrical Teflon mold with a 5 mm diameter and 10 mm height for characterization purposes. The solvent/non-solvent mixture is allowed to evaporate in a fume hood for 30 minutes followed by vacuum-drying for an additional 24 hours. Scaffolds of 8 mm diameter and 2 mm thickness are also fabricated for in vitro cell studies. Tubular scaffolds of 15×5 mm are fabricated to accommodate the size of rabbit ulnar non-union defect model for in vivo testing using the optimized parameters. Similarly CA microsphere scaffolds are fabricated using a solvent/non-solvent composition of acetonitrile:water at a ratio of 8:2. In contrast, control PLAGA (85:15 ratio) microspheres (850-600 μm) are sintered at 90° C. in a stainless steel mold for 2 h.

### 3-D Biomimetic Scaffolds—Nanofiber Surface Functionalization

Microspheres, e.g. EC microspheres, of a chosen diameter range are mixed with a solvent/non-solvent composition of 3:1 ratio of acetone:water to produce a slurry. The resulting slurry is placed in a cylindrical Teflon mold with a 5 mm diameter and 10 mm height for characterization purposes. The solvent/non-solvent mixture is allowed to evaporate in a fume hood for 30 minutes followed by vacuum-drying for an additional 24 hours. Scaffolds of 8 mm diameter and 2 mm thickness are also fabricated for in vitro cell studies. Tubular scaffolds of 15×5 mm are fabricated to accommodate the size of rabbit ulnar non-union defect model for in vivo testing using the optimized parameters. Similarly CA microsphere scaffolds are fabricated using a solvent/non-solvent composition of acetonitrile:water at a ratio of 8:2. In contrast, control PLAGA (85:15 ratio) microspheres (850-600 μm) are sintered at 90° C. in a stainless steel mold for 2 h. 3-D porous microsphere scaffolds are functionalized with collagen type I nanofibers to provide nanotopographical features. Surface functionalized nanofibers provide increased surface area as well as ECM like environment believed to favor cell recruitment and mineralization in vivo. Nanofiber functionalization is a carried out using an approach reported in the literature (Kim, et al., J. Biomed. Mater. Res. A (2005) 75: 629-638). In brief, collagen type I is dissolved in 50 mM acetic acid to produce a final concentration of 1% w/v. 3-D microsphere scaffolds are incubated with a reconstituted collagen solution in phosphate-buffered saline (PBS) at a concentration of 0.2% w/v at 37° C. for 24 h. These nanofiber functionalized scaffolds are washed with deionized water and kept desiccated until further use.

Scaffold surfaces are decorated with nanofibers of ECM (extracellular matrix) components termed as functionalization. This surface functionalization is achieved thorough physical as well as chemical modification protocols.

### Physical Functionalization:

Using the functionalization platform described in the previous step it is also possible to deposit nanofibers with other ECM proteins such as vitronectin, fibronectin and laminin to enhance in vitro cell attachment and in vivo performance. This process is quite similar to coating (physical deposition) scaffolds with nanofiber structures.

### Chemical Functionalization:

Alternatively ECM components, namely collagen type I, vitronectin, fibronectin and laminin, can be covalently attached to the scaffold surface via carbodiimide chemistry. In brief, hydroxyl and/or carboxylic groups on the polysaccharide scaffolds are activated by incubating scaffolds in aqueous solution of 1-ethyl-3-(3-dimethylaminopropyl) carbodiimide hydrochloride (EDC) and with N-hydroxysuccinimide (NHS) between pH 4-6 at room temperature. ECM components of know concentrations react with the previously activated OH/COOH groups via amine groups forming amide bonds. After the completion of the functionalization (typically 6-8 h) reaction scaffolds are washed with deionized water and kept desiccated until further use.

### Polysaccharide Scaffolds for Soft Tissue Regeneration

Fiber matrices of cellulose acetate or ethyl cellulose are produced by the process of electrospinning. In brief, 1.5 g of ethyl cellulose is dissolved in 10 mL solvent mixture of Tetrahydrofuran:Acetone (7:3 vol/vol). This solution is electrospun to produce fiber matrices. In a representative electrospinning experiment we use the following optimized electrospinning parameters:polymer solution flow rate 2 mL/h, 10 kV applied voltage, and 10 cm working distance and ambient conditions. Resultant fibers exhibit bead-free fiber morphology and the fiber diameters range from 300 nm-2000 nm. By increasing the polymer concentration it is possible to increase fiber diameter up to 5000 nm. Cellulose acetate fibers are also produced using the similar electrospinning parameters. Cellulose acetate is dissolved either in a solvent mixture of Acetic acid:Water (75:25 vol/vol) or Dimethylacetamide:Acetone (1:2 vol/vol) prior to electrospinning Electrospun fiber matrices closely mimic the structure and morphology of the native extracellular matrix (ECM). These polysaccharide matrices can be used as scaffolds for variety of soft tissue engineering applications such as skin, blood vessel, tendon/ligament, cardiac patch, nerve and skeletal muscle. These fiber matrices can also be used as scaffolds for cartilage repair and regeneration.

### Polysaccharide Scaffolds for Hard and Soft Tissue Interface

Integrating polysaccharide microsphere scaffolds with fiber scaffolds provides opportunities to work with tissue interfaces such as hard tissue (bone) and soft tissue (tendon/ligament/skeletal muscle). Such hybrid scaffolds can be fabricated by sintering microsphere scaffolds and fibers matrices using a similar solvent/non-solvent approach. For example, a Teflon mold is filled with the microspheres and one end of fiber scaffold is inserted into the same mold. Then a known quantity of a solvent system having an organic fraction and aqueous fraction composition is added to sinter microspheres and the inserted end of fiber matrix. Microsphere part of the hybrid scaffold can be used for repair and regeneration of hard tissue and fiber matrix for soft tissue.

### Evaluating Proliferation, Differentiation and Deposition of a Mineralized Matrix of Primary Human Osteoblast Cells Cultured on Composite Microsphere Scaffolds

The composite scaffold in vitro performance is evaluated by culturing primary human osteoblasts for up to 28 days. Cellular constructs are analyzed for adhesion, proliferation, mineralization as well as gene expression at different time intervals of 1, 3, 7, 14, 21 and 28 days post-seeding.

### Cell Culture

3-D microsphere scaffolds are incubated with 2 mL of DMEM supplemented with 10% FBS and 1% P/S in a 24 well plate at 37° C. in a humidified atmosphere. After 2 hours of incubation, the media is removed from the wells. Fifty thousand human osteoblasts from the subcultures are plated onto the scaffolds. Constructs are cultured at 37° C./5% CO₂ in mineralization medium, which consists of DMEM supplemented with 10% FBS, 1% P/S, 50 μg/mL ascorbate and 10 mM β-glycerophosphate. The culture media are changed twice a week.

### Cell Adhesion

At different time intervals on 1, 3 7, 14, 21 and 28 days cell seeded scaffolds are rinsed with PBS three times, soaked in 1% glutaraldehyde for 1 hour followed by 3% glutaraldehyde for 24 hours to fix the samples. After fixation these scaffolds (n=2 each group) are washed with deionized water, dried and imaged with SEM.

### Cell Viability

Viability of cells on 3-D composite microsphere scaffolds are imaged using a live/dead cell viability kit (Molecular Probes, L-3224). In brief, calcein AM enters live cells and reacts with intracellular esterase to produce a bright green fluorescence, while ethidium homodimer-1 enters only dead cells with damaged membranes and produces a bright red fluorescence upon binding to nucleic acids. 3-D composite microsphere scaffolds (n=2 each group) are imaged on 3, 7, 14 and 28 days using a BioRad Radiance 2100 Multiphoton/Laser Scanning Confocal Microscope (LSCM).

### Cell Proliferation

Cell proliferation on the composite surface is determined at 1, 3, 7, 14, 21 and 28 days post-seeding by quantification of the DNA concentration (n=4 each group). At the predetermined time points, the matrices are washed with PBS solution and the cells lysed with 1 mL of 1% triton X-100 (Bio-Rad, CA). Two freeze-thaw cycles are performed to ensure cell lysis. The cell lysate is collected and stored in −70° C. until analysis. The DNA concentration is determined using the Picogreen dsDNA assay (Molecular Probes, OR). The DNA concentration is measured as fluorescence using a Tecan UV spectrophotometer [Spectro Flour Plus, F129005, USA] at an emission and excitation wavelength of 485 nm and 535 nm respectively. The cell number is determined using a standard curve from known cell numbers.

### Alkaline Phosphatase Activity

The phenotypic bone marker, alkaline phosphatase, is determined at 1, 3, 7, 14, 21 and 28 days post seeding using an alkaline phosphatase substrate kit (Bio-Rad, CA). The cell lysate obtained from the DNA assay is used to evaluate alkaline phosphatase activity. Briefly, 100 μL of the cell lysate is added to 400-μL of p-nitrophenylphosphate substrate and the solution incubated at 37° C. for 30 minutes (n=4 each group). The reaction is stopped by the addition of 0.4M sodium hydroxide solutions. p-Nitrophenol is produced in the presence of alkaline phosphatase and the absorbance is measured at 410 nm using a Tecan UV spectrophotometer [Spectro Flour Plus, F129005, USA]. The change in the absorbance is a direct indication of the alkaline phosphatase activity. The absorbance is normalized based on the cell number obtained on each scaffold as determined from the DNA assay.

### Alizarin Red Calcium Quantification

Mineralized matrix synthesis by cells is analyzed with Alizarin Red staining method for calcium deposition. This quantitative technique is based on solubilizing the red matrix precipitate with cetylpyridinium chloride to yield a purple solution. In brief, samples are fixed with 70% ethanol at 4° C. for 1 h and then stained with 10% Alizarin Red (Sigma) solution for 10 min (n=4 each group). After washing five times with distilled water, the red matrix precipitate is solubilized in 10% cetylpyridinium chloride (Sigma), and the optical density of the solution is read at 562 nm with a spectrophotometer (Shimadzu). The calcium deposition is expressed as molar equivalent of CaCl₂ and normalized by the average number of cells per unit area as determined in companion proliferation studies.

### Real Time Reverse Transcription Polymerase Chain Reaction (RT-PCR)

In order to evaluate the gene expression of primary human osteoblasts cultured on 3-D composite microsphere scaffolds, RT-PCR is performed. Expression of type I collagen (T1C), ALP, osteocalcin (OCN), and osteopontin (OPN) is evaluated (n=4 each group). At predetermined time points of 3, 7, 14, 21 and 28 days, scaffolds are washed with PBS and the total RNA from the cells are isolated using Trizol following the procedure described by the manufacturer (Gibco BRL, 11596-026). The primers are designed on the basis of published gene sequences (NCBI and Pubmed). The RNA is converted to c-DNA in a thermal cycler and the concentration of the different genes are determined using a real time RT-PCR (Applied Biosystems, CA). The quantitative evaluation of the gene expression is determined using the Delta-Delta method and compared to the housekeeping gene (GAPDH).

### Methods of Treatment

Methods of treating a patient in need of bone replacement are included herein. The patient may be a human patient or other mammal. The patient may be in need of bone replacement due to traumatic injury, bone cancer, birth defects, to help fusion between vertebrae, correct deformities, and provide structural support to an injured spine. In one embodiment the method includes surgical excising a section of damaged bond and implanting the bone scaffold of sintered polysaccharide microspheres. The scaffold may be held in place with external stabilization devices, for example surgical pins, plates or screws. Depending on where the scaffold implantation is located and the size of the graft, an additional blood supply may be required. For these types of grafts, extraction a section of blood vessels from another part of the patient's body or from a donor and implantation along the implanted scaffold may be required. In certain embodiments the implanted scaffold additionally includes growth factors and/or collagen.

The bone replacement scaffolds described herein are also useful for replacing cartilage. When used for cartilage replacement the density and fibrous character of the scaffolds used should be adjusted to more closely match that of cartilage. For use in cartilage applications a more fibrous scaffold with higher density of packing than the bone replacement scaffold is required. Methods of adjusting the fibrous nature and density of the microsphere scaffolds disclosed herein as needed for particular applications by changing the size of microspheres used, the method of making the microspheres, and the sintering solvent are included herein.

### Methods of Making Bone Replacement Scaffolds

Also included herein is a method of making a scaffold for bone or cartilage replacement comprising providing a plurality of polysaccharide microspheres; providing a solvent system having an organic solvent fraction and an aqueous fraction; mixing the polysaccharide microspheres and the solvent system to form a slurry; molding the slurry to form a scaffold; and removing the solvent fraction from the scaffold. In this method, polysaccharide microspheres include ethyl cellulose microspheres or cellulose acetate microspheres. In certain embodiments the method of making a scaffold for bone or cartilage replacement additionally comprises one or more of the following (i) autoclaving the molded scaffold; (ii) having one or more antibiotics, antibacterial agents, or growth factors also present in the slurry; (iii) incubating the scaffold with a collagen solution after removal of the organic solvent fraction from the scaffold; (iv) a method as described herein in which the collagen is collagen type I, and the collagen solution is a 0.5% to 2.0% w/v collagen solution; (v) a method as described herein in which the scaffold has a compressive strength of at least 5 M Pa and a compressive modulus of at least 100 M Pa; and/or a method as described herein in which the scaffold has a pore diameter of 80 to 170 micrometers or pore volume of 25% to 75%.

All methods of making a scaffold for bone or cartilage replacement comprises any combination of steps or conditions (i) to (v) resulting in a usable and stable scaffold are included herein.

Scaffolds for bone or cartilage replacement comprising any combination of the features, ranges or limits described herein are included as embodiments so long as a usable bone or cartilage replacement scaffold results, unless the context clearly indicates that the limits or features cannot be combined.

### EXAMPLES

### Example 1

### Microsphere Fabrication

Cellulose acetate (CA) or ethyl cellulose microspheres (EC) are fabricated using an oil-in-water emulsion/solvent evaporation method. In brief, either CA or EC is dissolved in a binary solvent composition of methylene chloride:acetone (9:1) at 20% (w/v). The resulting polymer solution is slowly poured into a 1% (w/v) polyvinyl alcohol aqueous solution stirring at 250 rpm. The solvent is allowed to evaporate overnight at room temperature under constant stirring. The microspheres are collected by vacuum filtration and washed with distilled water. Microspheres are sieved and separated into different sizes based on their diameter for scaffold fabrication. Three different diameters namely >1180, 1180-850, and 850-600 μm were chosen for use in microsphere scaffolds.

### Example 2

### Scaffold Fabrication Using Solvent/Non-Solvent Sintering

It was necessary to identify a proper solvent/non-solvent composition for each polymer at which only the microsphere surface turns rubbery to facilitate bonding with the adjacent microspheres. After several trials a solvent/non-solvent composition of 3:1 ratio of acetone:water was found to be suitable for ethylcellulose (EC) microsphere sintering while 8:2 ratio of acetonitrile:water for cellulose acetate (CA). The sieved microspheres were mixed with sintering solvent and the mixture was vortexed for five seconds. The resulting slurry was placed in a cylindrical Teflon mold with a 5 mm diameter and 10 mm height. The solvent/non-solvent mixture was allowed to evaporate in a fume hood for 30 minutes followed by vacuum-drying for an additional 24 hours. Scaffolds of 8 mm diameter and 2 mm thickness were also fabricated for in vitro cell studies. In contrast control poly(lactide-co-glycolide) (PLAGA) sintered microsphere matrices were fabricated by heating 850-600 μm in diameter microspheres at 90° C. in a stainless steel mold for 2 h. Thus formed scaffolds were named as EC-600, EC-850, EC-1180 and PLAGA (control) based on the microsphere diameter. In general, to create the microsphere slurry a minimum amount of solvent-non-solvent composition was used just to wet the microsphere surfaces. Alternatively Teflon molds were filled with selected microspheres and 100 μL of solvent/non-solvent composition was added to each scaffold. 100 μL of solvent/non-solvent composition is just sufficient enough to wet the microspheres in a mold of 5 mm diameter and 10 mm height.

### Example 3

### 3-D Sintered Microsphere Characterization

### Morphology

3-D Composite microsphere scaffold morphology is characterized by SEM. Cylindrical scaffolds (n=3) are coated with gold using a Hummer V sputtering system (Technics, Baltimore, Md.) for 5 min. Samples are visualized on a JSM 6400 (JOEL, Boston, Mass.) at 15-20 keV and a working distance of 39-48 cm.

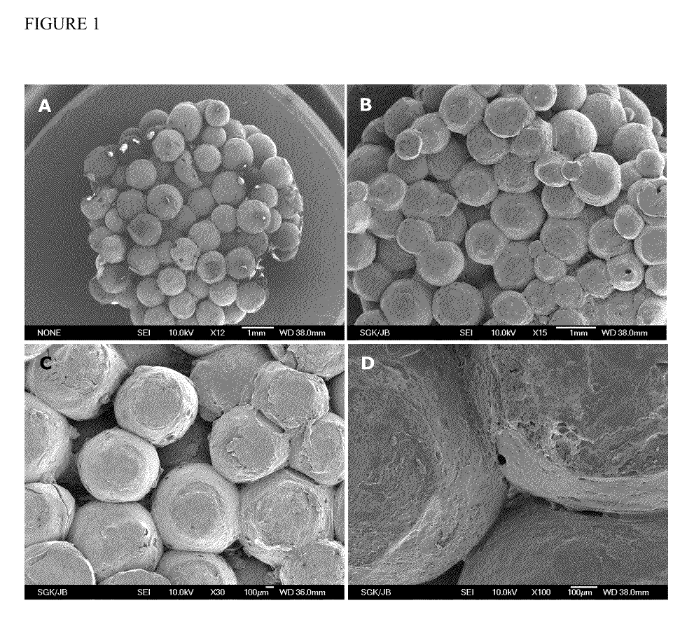

Scanning electron microscopy (SEM) is used to characterize the morphology of the individual microspheres and the corresponding scaffolds. FIG. 1 (A-D) illustrates interconnected pore structure, and the bonding between the adjacent microspheres. FIG. 1 shows SEM micrographs of solvent/non-solvent sintered (A) EC microspheres scaffold at 12× magnification and (B) same scaffold at a magnification of 15×. A-B illustrates the bonding between the adjacent microspheres and interconnected pore structure. FIG. 1 shows morphology of autoclave sterilized scaffold (C) at a magnification of 30× and (D) at 100× illustrate the intact scaffold structure after autoclave sterilization. Scaffolds retained their microsphere structure and interconnected porosity after autoclave sterilization. These scaffolds present the ideal structural and morphological features needed for bone tissue engineering applications.

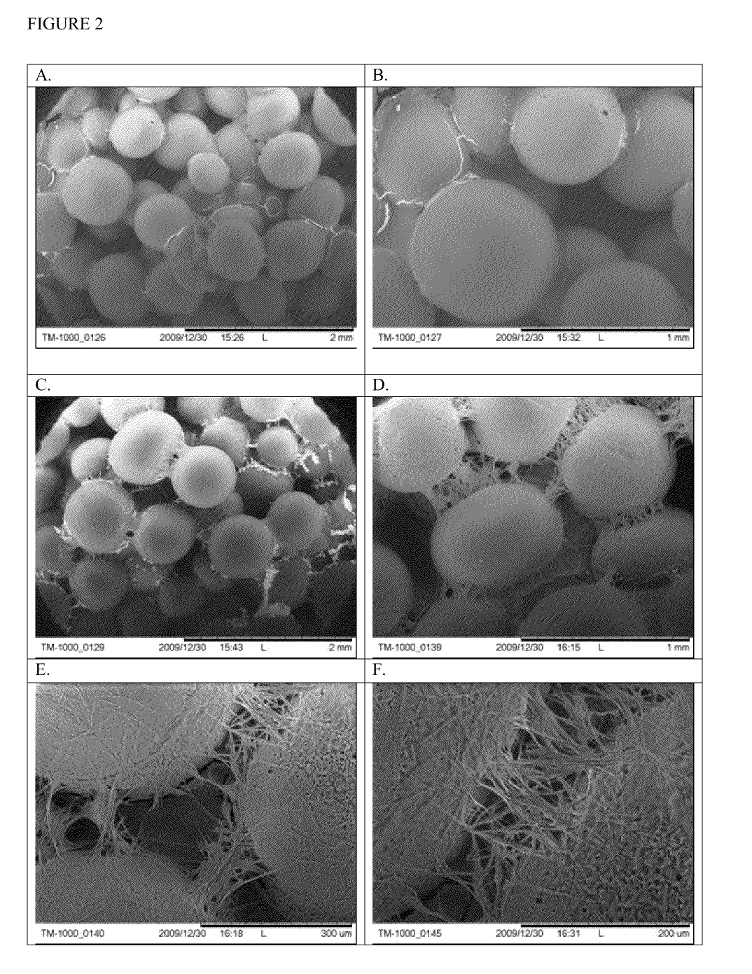

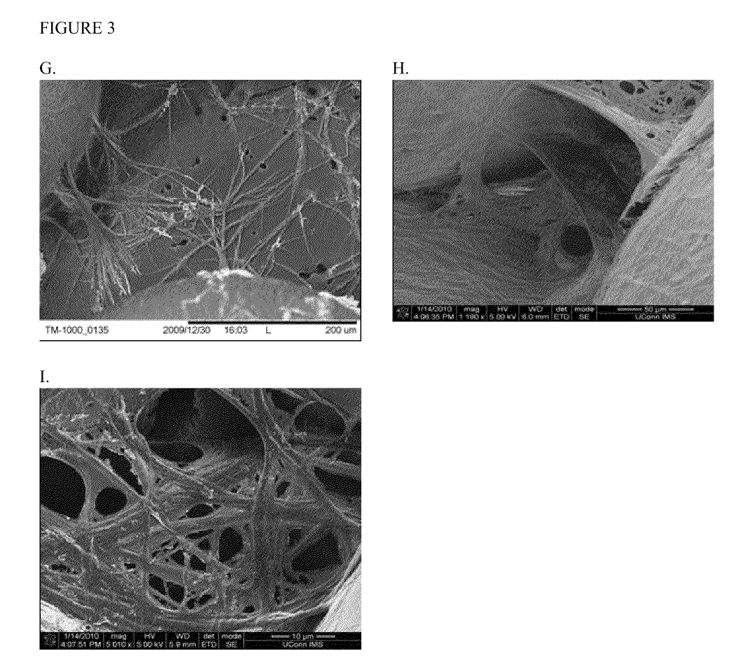

FIG. 2 represents SEM micrographs of cellulose acetate (CA) porous grafts functionalized with collagen nanofibers, where (A and B) are CA control grafts without functionalization at lower and higher magnification. Micrographs C-F are collagen functionalized CA grafts at various magnifications and depths of the scaffold structure. Micrographs C-F represents graft surface indicating individual microsphere having nanofiber functionalization and nanofibers extending throughout the structure. FIG. 3 shows micrographs of Cellulose Acetate porous grafts functionalized with collagen nanofibers. Micrographs G-I represents the surfaces of the grafts at various depths after breaking scaffold randomly. Micrographs H and I were recorded at high magnification using FE-SEM (Field Emission Scanning Electron Microscopy) after coating samples with Au/Pd. All other micrographs were recorded on Environmental SEM. SEM micrographs reveal the presence of collagen nanofibers on Cellulose Acetate individual particles at all the depths.

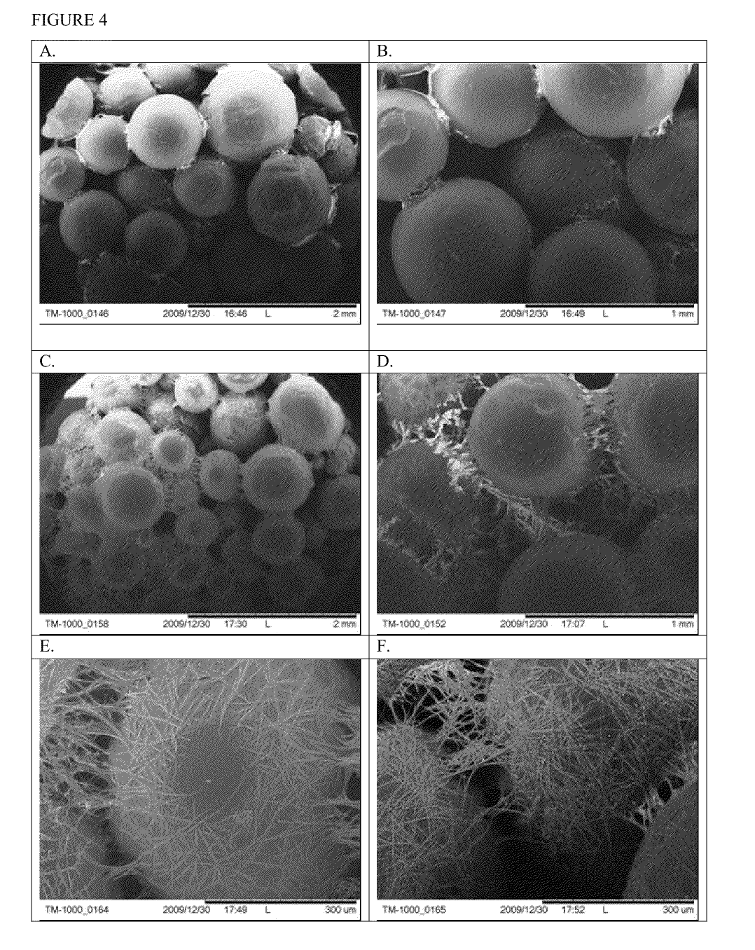

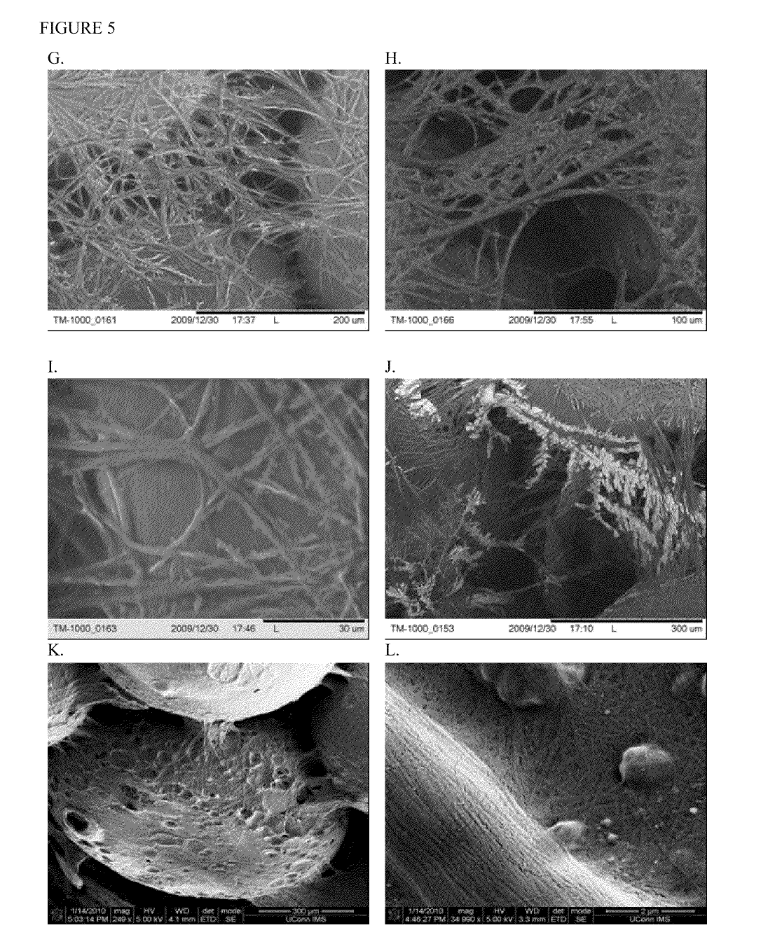

FIGS. 4 and 5 are SEM micrographs of Ethyl Cellulose (EC) porous grafts functionalized with collagen nanofibers, where (A and B) are EC control grafts without functionalization at lower and higher magnification. Micrographs C-L are collagen functionalized EC grafts at various magnifications and depths of the scaffold structure. Micrographs C-I represents graft surface indicating individual microsphere having nanofiber functionalization and nanofibers extending throughout the structure. Micrographs J-L represents the surfaces of the grafts at various depths after breaking scaffold randomly. Micrographs K and L were recorded at high magnification using FE-SEM after coating samples with Au/Pd. All other micrographs were recorded on Environmental SEM. SEM micrographs reveal the presence of collagen nanofibers on Cellulose Acetate individual particles at all the depths.

### Mechanical Properties

Earlier studies in the Laurencin laboratories reported a maximum compressive modulus of 363.4±46.6 MPa and the compressive strength of 5.8±2.0 MPa for PLAGA heat sintered microsphere (850-600 μm) scaffolds. In the fabrication of heat sintered PLAGA scaffolds there is a tradeoff between mechanical strength and porosity. For instance, either increasing the sintering temperature or time it is possible to produce more fused microsphere structures with improved mechanical properties while pore properties are compromised.

Tubular Scaffolds (n=6) at a length to diameter ratio of 2:1 (10×5 mm) are used for mechanical testing in compression. An Instron Testing Apparatus (model 5544; Instron, Canton, Mass.) is used at a ramp speed of 1 mm/min at ambient temperature, humidity and pressure until implant failure. Load and displacement are recorded to plot a stress versus strain curve. For each specimen, (1) compressive modulus (the slope of the linear region of the stress versus strain curve), (2) compressive strength (the magnitude of the maximum force applied divided by the original cross-sectional area), (3) maximum compressive load (the maximum force applied) and (4) the energy absorbed at failure (the area under the stress-strain curve at the point of failure) is calculated.

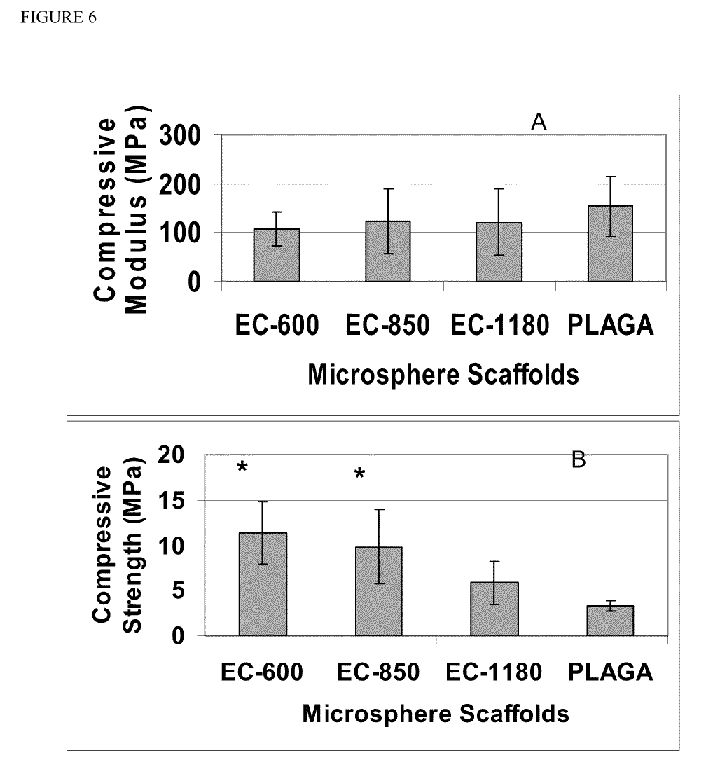

Cylindrical scaffolds (n=6) with 2:1 aspect ratio, required to meet ASTM standards for a cylinder, (10 mm length and 5 mm diameter), MW 65,000, were used for mechanical characterization. The compressive modulus was found to be 121.5±61.4 MPa and the compressive strength was 11.4±3.45 M Pa for ethyl cellulose microsphere scaffolds. However, PLAGA heat sintered microsphere scaffolds at the optimal sintering morphology and porosity showed a compressive modulus of 154.2±61.1 MPa and compressive strength of 3.3±0.61 MPa. (See FIG. 6.) These values are in the range of human trabecular bone for both the scaffolds. Because of higher compressive strength, cellulose scaffolds are stronger than PLAGA scaffolds for bone healing applications at load bearing sites. FIG. 6 (A) represents the variation of compressive modulus and 2(B) compressive strength with microsphere diameter.

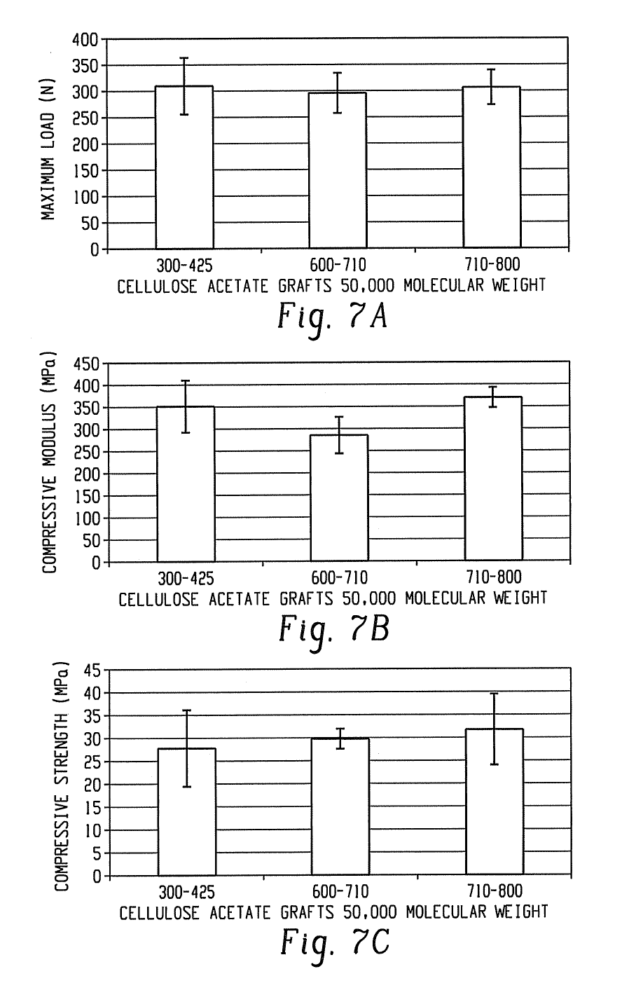

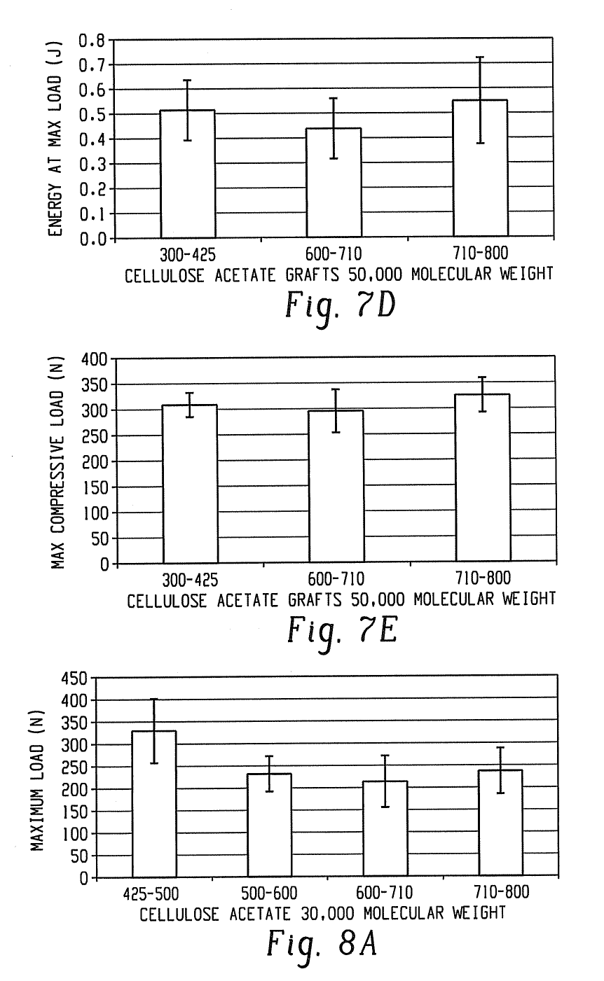

The mechanical properties of cellulose acetate porous grafts of molecular weight 50,000 fabricated by sintering particles in the range of 300-425, 600-710 and 710-800 microns are displayed in FIG. 7. In each of FIGS. 7-9 grafts measured 5×10 mm in dimension and were subjected for mechanical compression at a speed of 2 mm per min until failure. Graphs represent (A) Maximum Load (N) grafts can withstand, (B) Compressive modulus (MPa), (C) Compressive Strength (MPa). (D) Energy at Failure (J) and Maximum Compressive Load (N). Higher molecular weight cellulose acetate particles impart better mechanical properties compared to lower molecular weight cellulose acetate particles. Maximum load, compressive modulus, compressive strength, and maximum compressive load values for the cellulose acetate grafts are higher than Polyester PLAGA (85:15 or 80:20) grafts constructed by sintering micro particles. Cellulose acetate porous grafts of molecular weight 50,000 exhibited a compressive modulus of 290-350 MPa, a compressive strength of 25 MPa to 31 MPa, a maximum compressive load of 300-325N, and a maximum load of about 300 N.

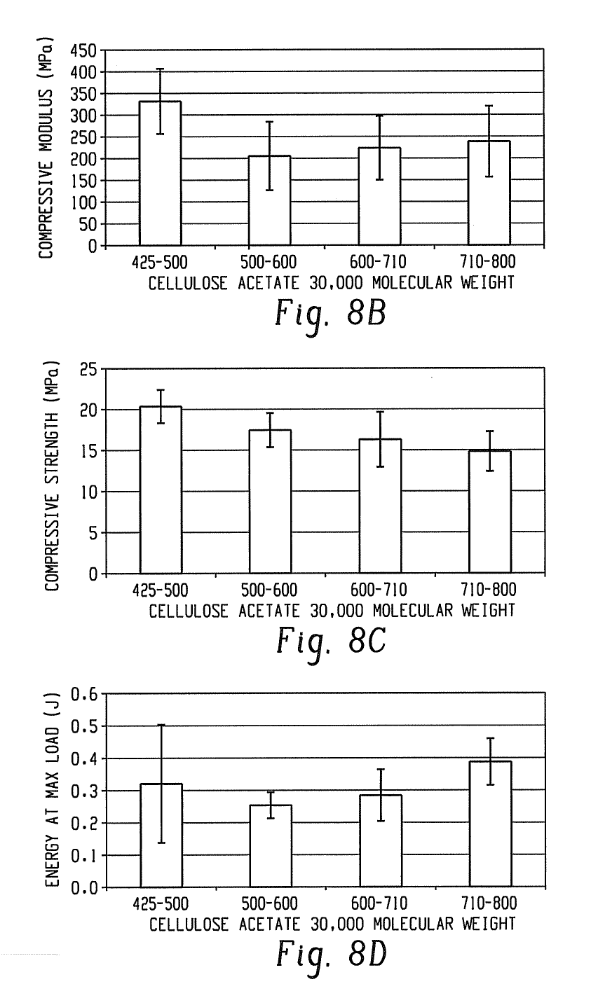

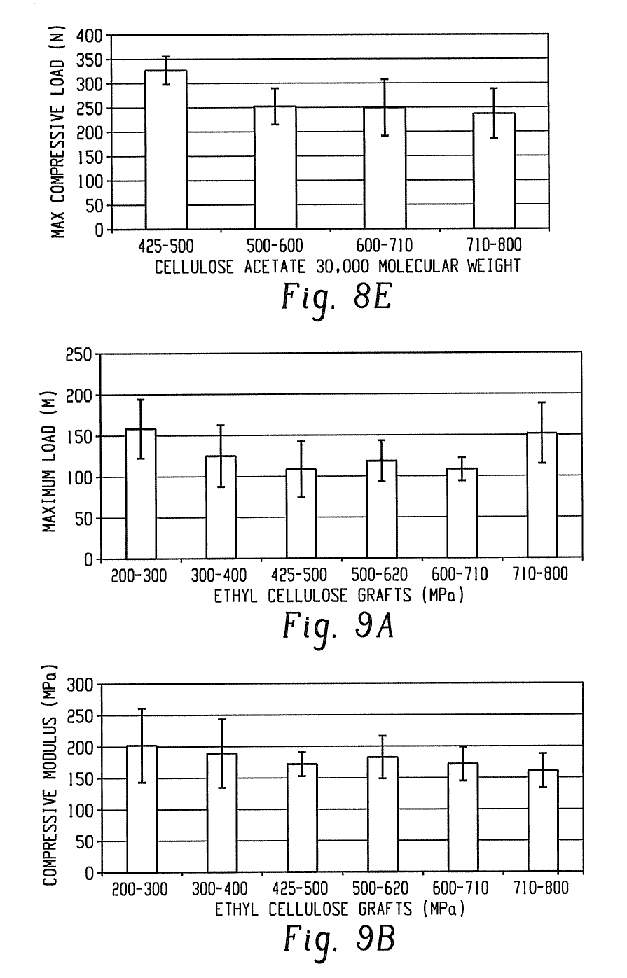

The mechanical properties of cellulose acetate porous grafts of molecular weight 30,000 fabricated by sintering particles in the range of 300-425, 600-710 and 710-800 microns are displayed in FIG. 8. Lower molecular cellulose acetate particles impart lower mechanical properties compared to higher molecular weight cellulose acetate particles. Ethyl cellulose porous grafts of molecular weight 30,000 exhibited a compressive modulus of 210-340 MPa, a compressive strength of 15 MPa to 21 MPa, a maximum compressive load of 240-330N, and a maximum load of 200 to 330 N.

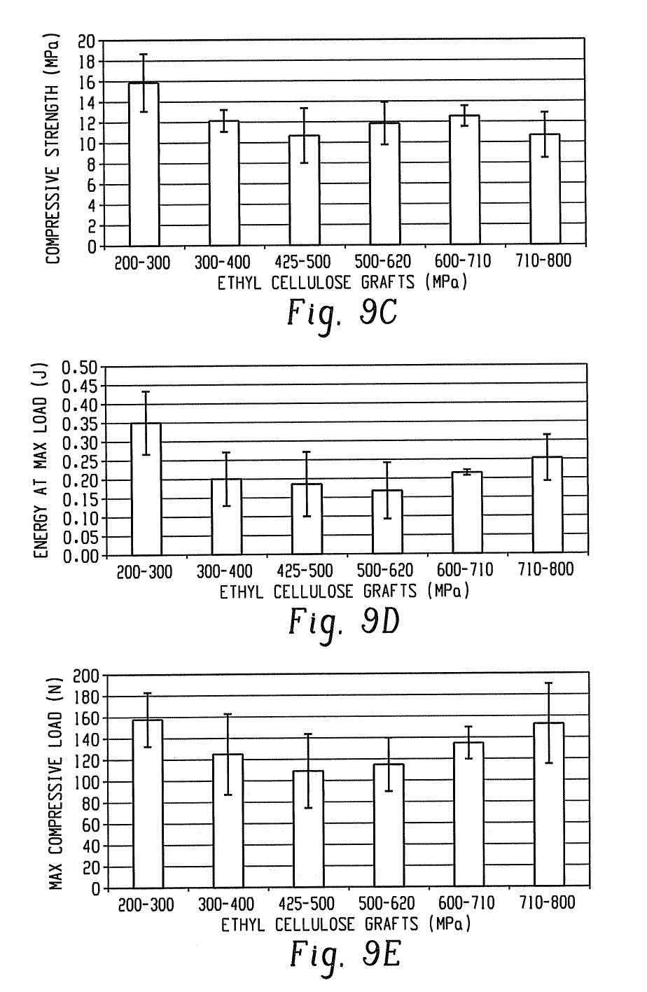

The mechanical properties of ethyl cellulose porous grafts of molecular weight 30,000 fabricated by sintering particles in the range of 200-300, 300-400, 425, 500, 500-620, 600-710 and 710-800 microns are shown in FIG. 9. Ethyl Cellulose grafts showed lower mechanical properties, lower strength, compressive modulus and load, compared to cellulose acetate grafts of similar molecular weight. Ethyl cellulose porous grafts of molecular weight 30,000 exhibited a compressive modulus of 160-200 MPa, a compressive strength of about 10.5 MPa to about 16 MPa, a maximum compressive load of 110-160N, and a maximum load of 110 to 160 N.

A bone replacement scaffold having a compressive strength of at least 5 MPa, or of at least 10 MPa is included herein. Further included herein is a bone replacement scaffold having a compressive modulus of at least 100 M Pa, or of at least 120 MPa, or of from about 80 MPa to about 400 MPa, or from about 100 MPa to about 350 MPa.

A bone replacement scaffold having a compressive strength of at least 10 MPa, or from about 10 MPa to about 35 MPa, or from about 15 MPa to about 30 MPa is included herein.

Also included is a bone replacement scaffold having a maximum compressive load of at least 100 N. A bone replacement scaffold having a maximum compressive load and a maximum load of about 100N to about 350 N is further included herein. Ethyl cellulose bone replacement scaffolds having a maximum compressive load and maximum load of about 100 N to about 170 N are provided herein. Also provided herein are cellulose acetate bone replacement scaffolds having a maximum compressive load of about 200 N to about 350 N.

### Porosity

Median pore diameter and percent porosity, defined as the ratio of scaffold void space to total scaffold volume, is determined using mercury intrusion porosimetry (Micromeritics Autopore III porosimeter; Micromeritics, Norcross, Ga.). Tubular scaffolds (8 mm diameter and 2 mm thickness, n=6) are placed in a 5-mL penetrometer, subjected to a vacuum of 0.5 psia, and infused with mercury to a maximum of 50 psia. Quantitative results are obtained using Micromeretics Software, which calculates the pore diameter D in relation to the external pressure P applied to force non-wetting liquid mercury into the pores. This is accomplished using the Washburn equation:

PD=−4Y cos θ

Where P is the applied pressure, D is the diameter, Y is the surface tension of mercury (0.48 N m⁻¹), and θ is the contact angle between the mercury and the pore wall. The measured median pore diameter, the pore volume and porosity were determined to be in the range of 155±10.2-103.2±8.1 μm, 63.43±11.51-29.25±5.6% and 29.59±6.85-17.24±3% respectively for the cellulose scaffolds. These values are in agreement with earlier reports characterizing PLAGA scaffolds.

In certain embodiments the polysaccharide microspheres have a pore volume of less than about 250 microns, or of less than about 200 microns, or of about 200 microns to about 50 microns, or of about 80 to about 170 microns, or of about 100 microns to about 160 microns. In certain embodiments the polysaccharide microspheres have a pore volume of less than about 80%, or of less than about 75% and more than about 10%, or of less than about 75% and more than about 25%, or of about 50%.

### Scaffold Degradation

Composite microsphere scaffolds are subjected for degradation in simulated body conditions at 37° C. One set of scaffolds is also subjected to cellulose enzyme catalyzed degradation under similar conditions. Changes in molecular weight and net scaffold weight loss over the different time points are measured. To measure changes in molecular weight, tubular microsphere scaffolds (n=3) are dissolved in tetrahydrofuran (THF) at a concentration of 1% (w/v). The solution is filtered through a 0.45μ polypropylene filter and analyzed using gel permeation chromatography (model 1100; Hewlett Packard) equipped with a Zorbax PSM 300S column preheated to 40° C. THF is used as the mobile phase at a flow rate of 1 mL/min. Polystyrene standards (Polymer Laboratories, Amherst, Mass.) are used for calibration.

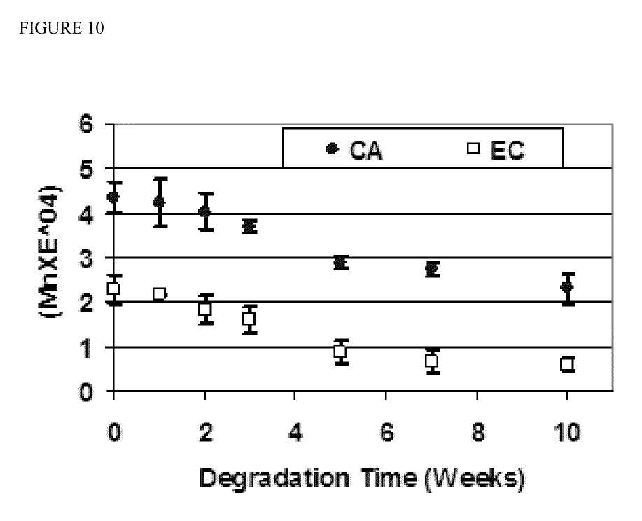

A set of 6 scaffolds of both CA and EC are subjected for degradation in acetate buffer solution at 37° C. with constant agitation for 10 weeks. Degradation patterns for both EC and CA scaffolds are presented in FIG. 10 show a decreasing trend in number average (Mn) molecular weight.

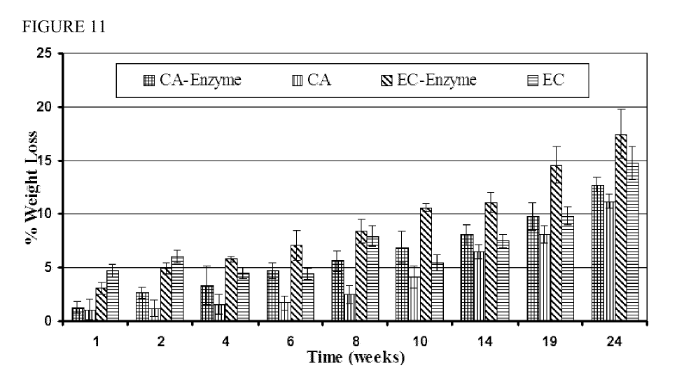

Cellulose Acetate (CA) and Ethyl Cellulose (EC) porous grafts measuring 4×8 mm in dimension were subjected for degradation for a period of 24 weeks at 37° C. in the presence and absence of cellulase enzyme (2 wt % in phosphate buffer (PBS) at pH 7.4) (FIG. 11). Degradation media was changed every alternate week with either PBS or cellulase enzyme in PBS. Samples were collected at predetermined time points, washed and dried. Changes in the initial and final weights are reported as % weight loss. For each measurement a sample size of n=8 was considered. At all the time points Ethyl cellulose showed higher weight loss than Cellulose Acetate grafts due to hydrophilic nature of EC. Grafts incubated in cellulase enzyme solution showed higher weight loss at all time points for both the grafts. Both EC and CA lost between 12-18% of their original weight in presence and absence of the enzyme. Mechanical Performance of Cellulose Acetate Porous Grafts, molecular weight 30,000 over period of 24 weeks of degradation in Phosphate Buffer pH 7.4 at 37° C.

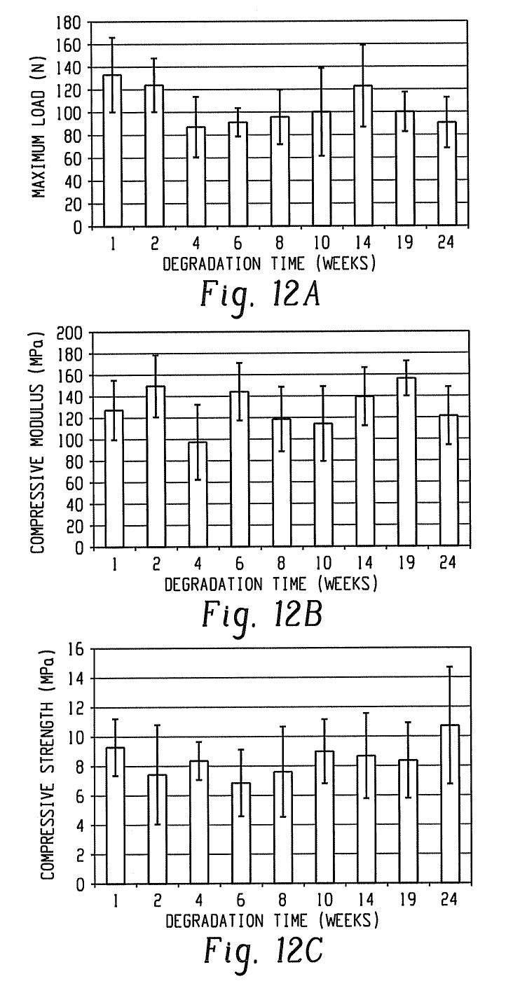

Cellulose Acetate Porous Grafts 30,000 of molecular weight in the particle size range of 600-700 micron were subjected for degradation in PBS (pH 7.4) at 37° C. to 24 weeks (FIG. 12). For each of FIGS. 12-15 grafts measured 5×10 mm in dimension and were subjected for mechanical compression at a speed of 2 mm per min until failure. Graphs (A) Maximum Load (N) grafts can withstand, (B) Compressive modulus (MPa), and (C) Compressive Strength (MPa) of the grafts over 24 weeks. These grafts remained intact during 24 weeks and mechanical properties were significantly lower than the original grafts. Changes in mechanical properties further confirm the scaffolds degradation.

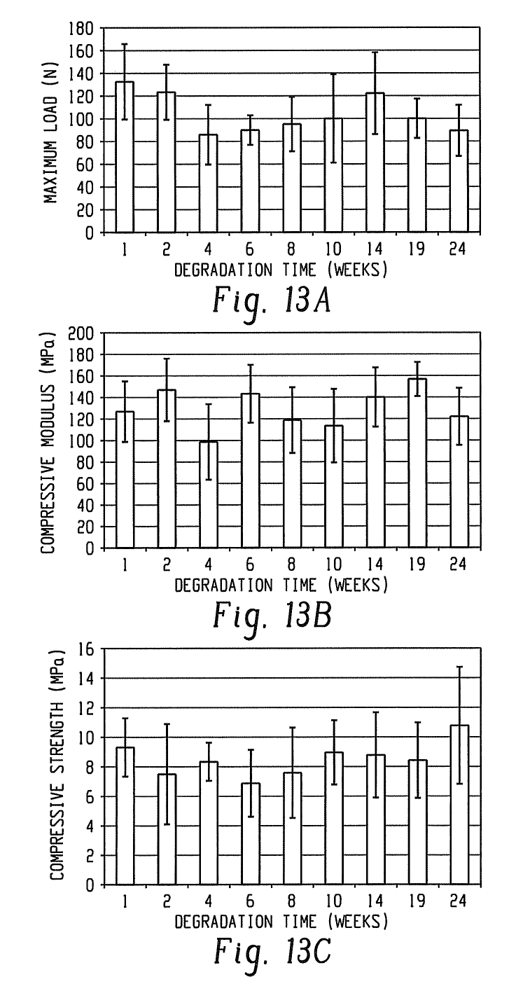

FIG. 13 shows the mechanical performance of cellulose acetate porous grafts, molecular weight 30,000 over period of 24 weeks of degradation in cellulase Enzyme (2 wt/vol % in Phosphate Buffer pH 7.4) at 37° C. As in FIG. 12 grafts remained intact during 24 weeks and mechanical properties were significantly lower than the original grafts but not different than PBS alone. Changes in mechanical properties further confirm the scaffolds' degradation.

Thus, cellulose acetate porous grafts exhibiting a decrease in maximum load of at least 20% in PBS or PBS plus cellulase enzyme over a 24 week period or of about 20% to about 40% in PBS or PBS plus cellulase enzyme over a 24 week period or of not more than 40% in PBS or PBS plus cellulase enzyme over a 24 week period are embodiments included herein. The invention provides cellulose acetate porous grafts exhibiting little or no decrease in compressive modulus or compressive strength in PBS over a 24 week period.

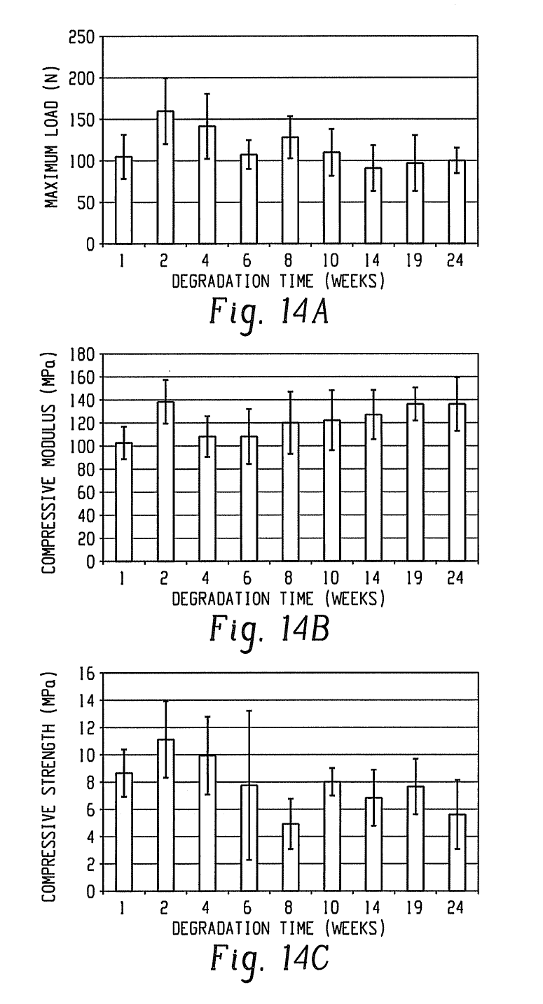

FIG. 14 shows the mechanical performance of ethyl cellulose porous grafts 30,000 of molecular weight in the particle size range of 600-700 micron when subjected to degradation in PBS (pH 7.4) at 37° C. to 24 weeks. As is FIG. 12 these grafts remained intact during 24 weeks and mechanical properties were significantly lower than the original grafts. Changes in mechanical properties further confirm the scaffolds' degradation.

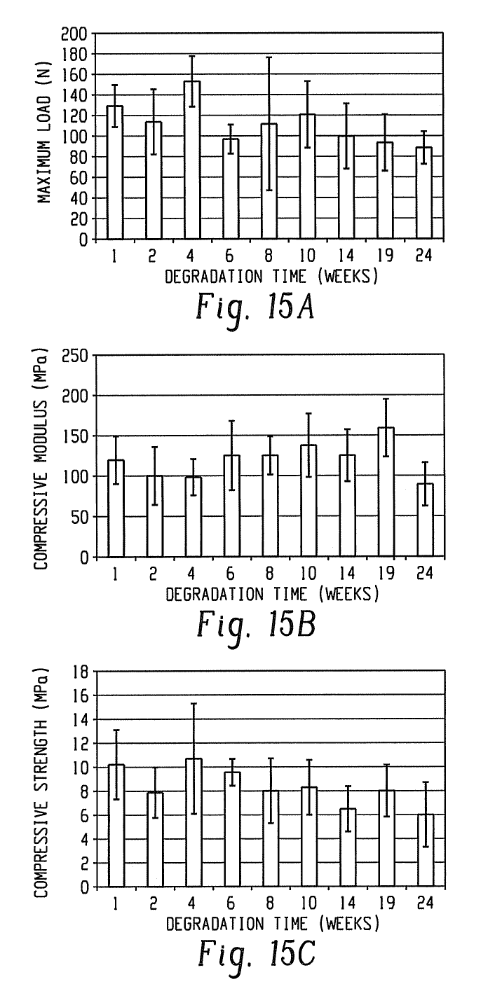

FIG. 15 shows ethyl cellulose porous grafts 30,000 of molecular weight in the particle size range of 600-700 micron were subjected for degradation in cellulase enzyme solution (2 Wt/Vol % in PBS (pH 7.4) at 37° C. to 24 weeks. As in FIG. 12 grafts remained intact during 24 weeks and mechanical properties were significantly lower than the original grafts but not different than PBS alone. Changes in mechanical properties further confirm the scaffolds' degradation.

Thus, the invention provides ethyl cellulose porous grafts exhibiting a decrease in maximum load of at least 20% in PBS or PBS plus cellulase enzyme over a 24 week period or of about 20% to about 40% in PBS or PBS plus cellulase enzyme over a 24 week period or of not more than 40% in PBS or PBS plus cellulase enzyme over a 24 week period. The invention provides ethyl cellulose porous grafts exhibiting little or no decrease in compressive modulus in PBS or PBS plus cellulase enzyme over a 24 week period and exhibiting at least 10% decrease in compressive strength in PBS or PBS plus cellulase enzyme over a 24 week period.

The invention also includes a porous bone graft exhibiting a percent weight loss of less that 20% in PBS over a 24 week period or from about 5% to about 20% in PBS over a 24 week period. In certain embodiments the porous bone graft is comprised of polysaccharide microspheres, such as cellulose acetate or ethyl cellulose microspheres.

### Example 4

### Bioactive Agent Delivery from the Scaffolds

The antibiotic gentamicin was used as a model drug to explore feasibility of bioactive agent incorporation during scaffold fabrication.

Gentamicin is encapsulated into the individual microspheres during microsphere fabrication. EC or CA polymer solutions are mixed with 5, 10 or 15% gentamicin (wt/wt ratio polymer dry weight:drug) and homogenized by vigorous mixing followed by sonication. This homogeneous polymer-drug solution is dispersed in aqueous media containing 1% PVA at an agitation speed of 250 rpm. Solvent is allowed to evaporate overnight and microspheres are isolated and washed as described in scaffold fabrication step. For illustrative purposes CA microspheres with 2 different drug loadings, namely 5 and 15% are presented.

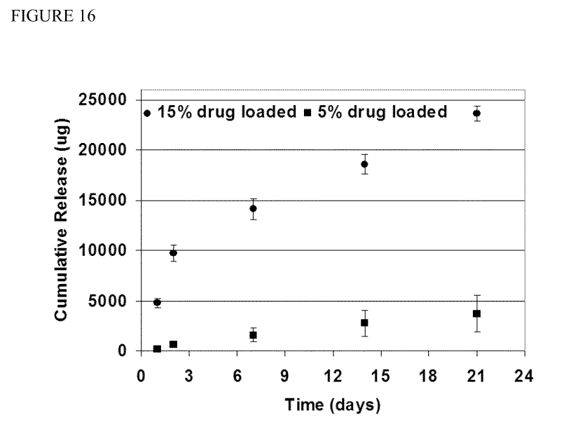

FIG. 16 presents a gentamicin release pattern over 21 days in phosphate buffer solution at pH 7.4 and 37° C. Gentamicin release followed a zero order release pattern for both drug loadings

### Example 5

### Cell Proliferation, Differentiation and Morphology on 3-D Sintered Microsphere Scaffolds

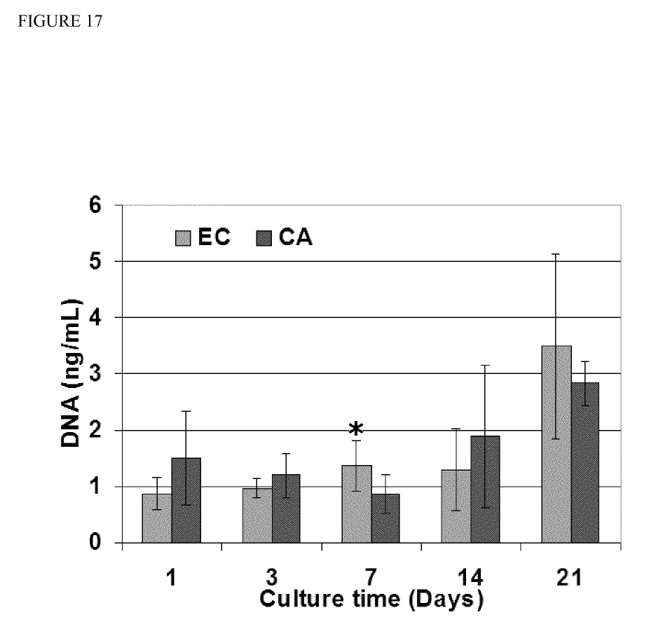

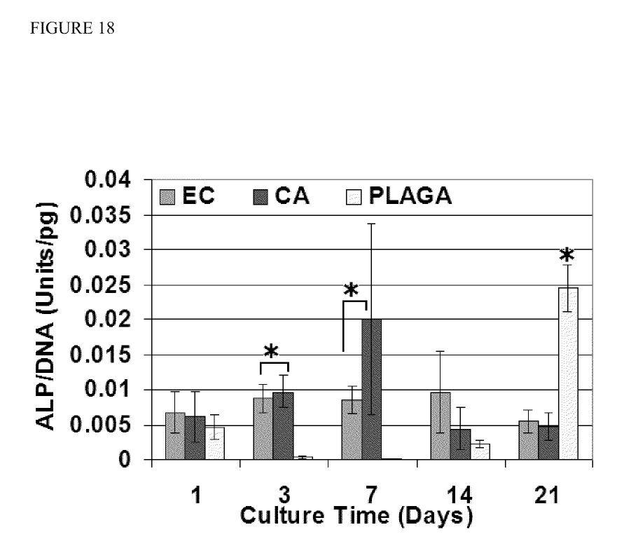

Scaffolds are seeded with 50,000 murine MC3T3E1 Subclone 4 preosteoblasts to evaluate cell proliferation alkaline phosphatase (ALP) expression and morphology. The scaffolds are then analyzed for double stranded DNA using a Pico Green Assay and analyzed for alkaline phosphatase activity using an alkaline phosphatase substrate assay (Sethuraman, et al., J. Biomed. Mater. Res. A. (2007) 82: 884-891, Hattori, et al., Cells Tissues Organs, (2004) 178: 2-12). MC3T3E1 preosteoblast proliferation is presented in FIG. 17 and alkaline phosphatase activity is presented in FIG. 18. Cells showed steady growth on polysaccharide scaffolds up to 21 days of culture time (FIG. 17). PLAGA control scaffolds exhibited DNA increasing at a greater rate than the polysaccharide scaffolds; whereas, ALP/DNA expression on polysaccharide scaffolds was higher at earlier time points as compared to PLAGA and demonstrated a peak in intensity at day 7 or 14, for CA and EC respectively (FIG. 6). These observations suggest a proliferative state of preosteoblasts on control PLAGA scaffolds up to 14 days; whereas, the rise and fall in the level of ALP/DNA expression on polysaccharide scaffolds suggests the preosteoblasts are progressing to express a mature osteoblast phenotype.

### Example 6

### Scaffold Mineralization

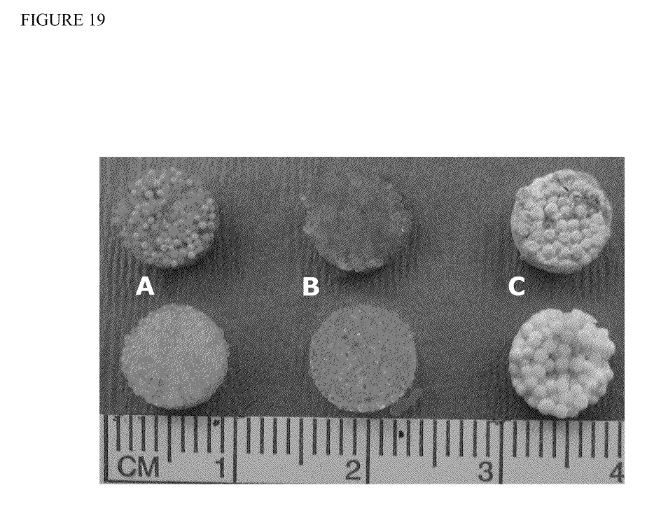

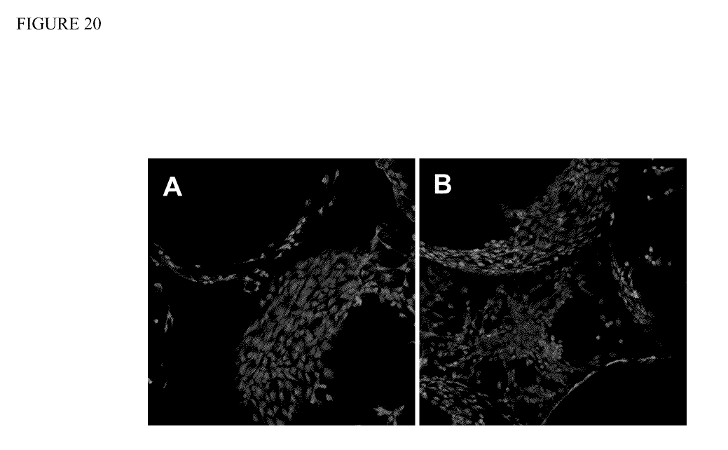

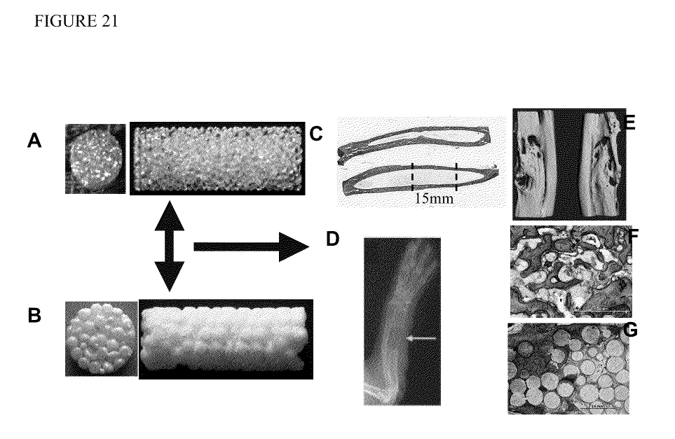

Mineralized matrix production by MC3T3-E1 preosteoblasts in osteogenic media at 7 days on the scaffolds is demonstrated using alizarin red staining of calcium deposits. Positive alizarin red staining (FIG. 19) confirmed the presence of a mineralized extracellular matrix after just 7 days in culture. FIG. 20 shows the cell morphology and survival on these novel polysaccharide scaffolds. Live cells appear as fluorescent green color and dead cells as fluorescent red. Osteoblasts survived on the CA and CA polysaccharide scaffolds throughout the culture period.

### Example 7

### In Vivo Evaluation of the 3-D Microsphere Scaffolds in a Critical Size Ulnar Defect Model

3-D porous mechanically competent microsphere scaffolds of naturally derived polymers are implanted in the rabbit ulnar critical size defect to evaluate the bone healing potential. Every 4 weeks, the rabbit fore limbs are x-rayed to determine the extent of healing. At 4 and 12 weeks, animals are sacrificed and the extent of mineralized tissue formation is quantified using micro-CT. Histological evaluation is performed by staining with Sanderson's rapid bone stain, and the mechanical properties of the defect site evaluated using compression testing.

### Ulnar Defect Model

The following animal study is performed in accordance with the Institutional Animal Care and Use committee regulations.

Adult New Zealand White rabbits (4-5 kg) are randomly divided into 5 groups: (1) 3-D microsphere scaffolds of CA (n=24), (2) 3-D microsphere scaffolds of EC (n=24), (3) 3-D microsphere scaffolds of PLAGA (control) (n=24), (4) 3-D composite microsphere scaffolds of CA (n=24) and (5) 3-D composite microsphere scaffolds of EC (n=24).

Rabbits are anesthetized through intramuscular injection of ketamine (50 mg/kg), xylazine (6 mg/kg), and acepromazine (1 mg/kg). Baytril (10 mg/kg) is administered prior to surgery. The right forelimb is shaved and sterilely prepped with betadine and 70% ethanol. The entire animal is covered with a sterile drape. A lateral incision approximately 2.5 cm is made and the tissues overlying the ulna dissected. A 1.5 cm segmental osteoperiosteal defect is created in the middle of the ulna using an oscillating saw. The radius is left intact for mechanical stability. The bony defect created is substituted with sintered microsphere scaffolds of similar dimension. The scaffold may be attached to the bone with surgical screws, plates and the like. However in this example the scaffold was simply placed at the defect site. The soft tissues are closed in layers over the scaffold, the incision is closed using Vicryl sutures, and the limb wrapped in a bandage. This suturing holds the scaffold in place without any external fixation. X-rays are obtained immediately post-operative and every 4 weeks thereafter. At 4 and 12 weeks, the animals are sedated with 1 mg/kg acepromazine and euthanized with a lethal dose of sodium pentobarbital (175 mg/kg). Samples are collected for microCT analysis, histological analysis, and mechanical testing in compression.

The outer skin is dissected from the forelimb and the remaining tissues left intact. The limb is harvested by further dissection at the elbow and wrist joints and transferred to a histological container filled with 1×PBS without Ca or Mg (samples for mechanical testing) or 10% neutral buffered formalin (samples for histology). Contralateral control limbs are prepared in the same manner. Specimens for mechanical testing are stored at −80° C. and histological specimens fixed at 4° C.

### MicroCT

The microCT (model viva CT 40; Scanco Medical, Bassersdorf, Switzerland) and accompanying analysis software are used to perform all image scanning, data processing, and analysis. Immediately prior to scanning, the limb is transferred to a 50 mL centrifuge tube containing 1×PBS. Each limb is scanned prior to subsequent mechanical testing or histology.

A control file was created to define the scanning parameters, which include source energy, sample size, and image resolution. The parameters selected for this study include a source voltage of 45 kVp and I of 175 μA. Standard instrument settings are used including Sigma_Gauss at 1.2, Support_Gauss at 2.0, Threshold_Seg. At 300, peel_iter_gobj at 0, and upper_threshold at 1000. The scan covers a 30 mm segment of the radius and ulna, centered at the defect site. The scan consists of 791 slices in 38 μm increments to create an image of 1024² voxels. Scan time is 55 minutes per sample.

From the initial scan, a contouring method is used to select a 15.20 mm segment of the ulna centered at the defect site for quantitative assessment of tissue formation on the exterior and interior of the scaffold. The contouring method is adjusted to select 5 mm diameter segments of the scaffold for tissue formation at the interior of the matrix. Direct measurements based on the selected contours determine the primary parameters tissue volume, bone volume, and relative bone volume.

### Biomechanical Testing

The forelimbs are slowly thawed at 4° C. overnight and equilibrated to room temperature over several hours (n=6 each group). The adhered tissue is removed and a mini rotary tool equipped with a saw blade is used to cut just above and below the proximal and distal bone-implant interface and radius. The radius and ulna segment is tested due to the result of radius-ulnar synostosis, which is commonly observed in critical size defects of the rabbit forelimb. During synostosis there is union of radius and ulna along the length of the interosseous membrane. Thus biomechanical testing of ulna alone becomes difficult due to which the entire segment was tested in compression using the Instron at ambient temperature and humidity at a ramp speed of 1 mm/min.

### Histological Staining

Forelimbs are fixed for several weeks in 10% formalin, dehydrated through a graded ethanol series, and processed using Spurr embedding medium (n=3 each group). The undecalcified are ground and 6 μm-thick sections cut for histochemical analysis. Sections are stained with Sanderson's rapid bone stain (Surgipath Medical Industries, Richmond, Ill.). Images are obtained using a digital camera attached to a Zeiss Axioskop 40 microscope in conjunction with PictureFrame software.

### Statistical Considerations

Experimental designs were based on statistical considerations to provide valid and meaningful conclusions. Scaffold design and characterization experiments were based on the previous studies and power analysis. For a α value of 0.05, a β value of 0.1, and a standard deviation from mean of 10%, the sample size was computed to be 6 for mechanical testing, 4 for porosimetry and 6 for degradation studies, for a power of 0.9. For in vitro cell studies power analysis provided a sample size of 4 for each group. One-Way Analysis of Variance (ANOVA) is used to compare the results between the groups. Two-way ANOVA is used to evaluate the cellular response at different time points. Tukey's tests are performed to determine the level of significance. Statistical significance for both cases is determined at p<0.05. For in vivo studies the sample size was computed from power analysis, by taking values from previous studies in Laurencin laboratories and the literature. Using the same set of parameters the sample size was computed to be 11 for a power of 0.9. We use a sample size of 12 for statistical significance.

What is claimed is:

1. A scaffold for bone or cartilage replacement, comprising at least 70 percent by weight sintered polysaccharide microspheres.

2. The scaffold of claim 1, where the sinter polysaccharide microspheres comprise derivatized cellulose microspheres.

3. The scaffold of claim 1, wherein the polysaccharide microspheres comprise ethyl cellulose microspheres and/or cellulose acetate microspheres.

4. The scaffold of claim 1, wherein the polysaccharide microspheres have a microsphere diameter of about 100 micrometers to about 1200 micrometers.

5. The scaffold of claim 4, wherein the polysaccharide microspheres have a microsphere diameter of about 650 to 850 micrometers.

6. The scaffold of claim 1, wherein the scaffold is autoclaved.

7. The scaffold of claim 1, wherein the scaffold additionally comprises an antibiotic, a growth factor, or a tissue response modifier.

8. The scaffold of claim 1, wherein the scaffold is functionalized with collagen nanofibers.

9. The scaffold of claim 1, wherein the scaffold has a compressive strength of at least 5 M Pa and a compressive modulus of at least 100 M Pa.

10. The scaffold of claim 1 having a pore diameter of 80 to 170 micrometers and/or pore volume of 25% to 75%.

11. A method of making a scaffold for bone or cartilage replacement comprising
providing a plurality of polysaccharide microspheres;
providing a solvent system having an organic solvent fraction and an aqueous fraction;
mixing the polysaccharide microspheres and the solvent system to form a slurry;
molding the slurry to form a scaffold; and
removing the solvent fraction from the scaffold.

12. The method of claim 11 wherein the polysaccharide microspheres include ethyl cellulose microspheres or cellulose acetate microspheres.

13. The method of claim 12, additionally comprising autoclaving the molded scaffold.

14. The method of claim 12, wherein one or more antibiotics, antibacterial agents, or growth factors is also present in the slurry.

15. The method of claim 12, additionally comprising incubating the scaffold with a collagen solution after removal of the organic solvent fraction from the scaffold.

16. The method of claim 15 wherein the collagen is collagen type I, and the collagen solution is a 0.5% to 2.0% w/v collagen solution.

17. The method of claim 12, wherein the scaffold has a compressive strength of at least 5 M Pa and a compressive modulus of at least 100 M Pa.

18. The method of claim 12, wherein the scaffold has a pore diameter of 80 to 170 micrometers and/or pore volume of 25% to 75%.

19. A method of treating a patient in need of bone repair, comprising implanting a bone replacement scaffold of claim 1 in the patient at a site of bone damage or bone deformity.

20. The method of claim 19, additionally comprising first excising damaged or deformed bone from the patient.

21. The scaffold of claim 7, wherein the scaffold comprises collagen nanofibers and two or more growth factors.

22. The scaffold of claim 21, wherein at the growth factors are VEGF and osteogenic factor (BMP-2) and the VEGF and BMP-2 are not released simultaneously from the scaffold.
